SEQUENCE LISTING (28 sequences)

SEQ ID NO: 1 - DNA, 1005 bp, Musa acuminata
atgggcaggggaagggtggagctacggaggatcgagaacaagatcaaccggcaggtcacgttcgcgaagcggaggaacggcctgctgaagaaggcctacgagctctccgtgctctgcgatgccgaggttgccgtcatcgtcttctccagccgcggcaagctctatgagttctgcagcggttccagcatgatgagaacacttgagaggtatcaaaagtgcagctatggagggtcagaaagcactatacaagcaaaggagaatcagttggttcaaagcagtcgtcaagagtacttaaaacttaaagcacgtctagaggctttacaaagatcacaaaggaatctccttggtgaggacttgggatcattaagtatcaaggagcttgattaccttgagaaacaactggacatgtcactgaaggaaattagatccacgaggacacaacaaatgcttgaccaactgaccgatcttcaaaggagggagcaattgctttgtgaagcgaacaagggtctcagaagaaggttggaggagagcagccatgctaatgggggacaattatgggaaaacagtgcccatccagcagctcagcagccacatggtgatgggttattctacccattggagtgtcaacccactccacagattggataccaacctgatcaaatgcctggcaccagtgtgagcacttatatgcctgcatggctggaatgatatgaccattctcttctgcagtcgtactgatcaaacttccacaagattaaaagtagtgaacttctggctgtgatgctgtattagattttctaatacataaactcataaactgatgaagccagtctataatgtctgcactttttttggggtatgtacttgaaagaaaggatgcccttctagaatgctgtttactagttgggaaacaattcttagtctaaatgaagactgattaatgattacacttcttagaggcatgtataataatctgccttttcacagtcaaaaaaaaaaaaaaaa

SEQ ID NO: 2 - DNA, 1004 bp, Musa acuminata
atggggagggggagagtggagctgaagcggatcgagaacaagatcaatcggcaggtaacgttcgcgaagcggaggaacggactgctcaagaaagcctacgagctttcggtgctctgcgatgctgaggtcgccctcatagtcttctccaaccgcgggaagctttacgagttctgcagcagctccagtatgctaaagacactggagaggtaccagaaatgcaactacggggcaccggaaacaaatataatatcaagggagatccaaactagccaacaggagtacttgaagctcaaggcacgtgttgaagccttgcaacgatcacaaagaaatctgctgggggaggatctggggccactcagcatcaaggagctcgagcaactggagcgccaactcgatgcatcgttgcgacaaatacgatcgacaaggacgcagtgcatgcttgaccaacttgctgatctccaaagaagggaacaaatgctctgtgaagctaataaagctctgaagataagaatggatgaaggaaaccaggccaatcagcaacaactgtgggatcctaatgctcatgctgtggcttactgccgccaccaaccacagccgcagggtgacgggttcttccaacccatagaatgcgaacctactcttcaaatcgggtatcatccagatcagatggcgattgctgcggcagcacctgggccgagtgtgagtagctacgtgccaggatggcttgcttgacagcaagataagaccggcatttctgggtgcctctaattatgtgttgtatgctagcgaggaatgctcctatttcttgtgctgatgattgcgtatataactggcaaacatgatgtcaattactgtcggatgatctatatgttattaatgacctgctataaagacctccttcggagggaggtagtcccaagcgtcgtatgtactcgaatgctatttggttctcctcctaagttgctgtcagctatgatgatttggtttcaaaaaaaaaaaaaaaa

SEQ ID NO: 3 - DNA, 18 bp, Musa acuminata
gcaaggatgccccaatgt

SEQ ID NO: 4 - DNA, 22 bp, Musa acuminata
agcaagacagttggttgtgcag

SEQ ID NO: 5 - DNA, 21 bp, Musa acuminata
gcgacgcatcattcaaatttc

SEQ ID NO: 6 - DNA, 21 bp, Musa acuminata
tccggaatcgaaccctaattc

SEQ ID NO: 7 - DNA, 53 bp, Musa acuminata
ggggacaagtttgtacaaaaaagcaggctaaggaatctccttggtgaggactt

SEQ ID NO: 8 - DNA, 53 bp, Musa acuminata
ggggaccactttgtacaagaaagctgggtaatctgtggagtgggttgacactc

SEQ ID NO: 9 - DNA, 53 bp, Musa acuminata
ggggacaagtttgtacaaaaaagcaggctggaaaccaggccaatcagcaacaa

SEQ ID NO: 10 - DNA, 53 bp, Musa acuminata
ggggaccactttgtacaagaaagctgggtcgcaatcatcagcacaagaaatag

SEQ ID NO: 11 - DNA, 28 bp, Musa acuminata
ctgctctcgagcgcaatcatcagcacaa

SEQ ID NO: 12 - DNA, 28 bp, Musa acuminata
tggcggaattcggaaaccaggccaatca

SEQ ID NO: 13 - DNA, 24 bp, Musa acuminata
atcattgatcttacatttggattg

SEQ ID NO: 14 - DNA, 24 bp, Musa acuminata
gtctcagaagaaggttggaggaga

SEQ ID NO: 15 - DNA, 24 bp, Musa acuminata
atcattgatcttacatttggattg

SEQ ID NO: 16 - DNA, 21 bp, Musa acuminata
cagggtgacgggttcttccaa

SEQ ID NO: 17 - DNA, 21 bp, Musa acuminata
gtggattgatgtgacatctcc

SEQ ID NO: 18 - DNA, 28 bp, Musa acuminata
tggcggaattcggaaaccaggccaatca

SEQ ID NO: 19 - DNA, 23 bp, Musa acuminata
acaactggacatgtcactgaagg

SEQ ID NO: 20 - DNA, 20 bp, Musa acuminata
gctggatgggcactgttttc

SEQ ID NO: 21 - DNA, 20 bp, Musa acuminata
caggtgacgggttcttccaa

SEQ ID NO: 22 - DNA, 24 bp, Musa acuminata
cgatttgaagagtaggttcgcatt

SEQ ID NO: 23 - DNA, 21 bp, Musa acuminata
ttgatcctggagcagatggaa

SEQ ID NO: 24 - DNA, 22 bp, Musa acuminata
gctttcaaggtggcaccttcta

SEQ ID NO: 25 - DNA, 22 bp, Musa acuminata
tcccaacactcatgctgtagct

SEQ ID NO: 26 - DNA, 20 bp, Musa acuminata
cgccatttgatctggatggt

SEQ ID NO: 27 - DNA, 22 bp, Musa acuminata
ccattgtggacgtcaattctca

SEQ ID NO: 28 - DNA, 19 bp, Musa acuminata
aaagcgtcgcccatcaagt

(19) United States
(12) Patent Application Publication
(10) Pub. No.: US 2013/0036515 A1
(43) Pub. Date: Feb. 7, 2013

(54) Banana MADS-Box Genes for Banana Ripening Control

(71) Applicants: James J. Giovannoni, Ithaca, NY (US); Haya Friedman, Rehorot (IL); Julia Vrebalov, Ithaca, NY (US); Tomer Elitzur, Doar Na Mercaz (IL)

(72) Inventors: James J. Giovannoni, Ithaca, NY (US); Haya Friedman, Rehorot (IL); Julia Vrebalov, Ithaca, NY (US); Tomer Elitzur, Doar Na Mercaz (IL)

(21) Appl. No.: 13/565,973

(22) Filed: Aug. 3, 2012

Related U.S. Application Data
(60) Provisional application No. 61/515,351, filed on Aug. 5, 2011.

(51) Int. Cl. A01H 5/00; A01H 5/08; C12N 15/82

(52) U.S. Cl. 800/286; 800/278; 800/298; 435/320.1

(57) ABSTRACT

The ripening of banana fruit may be delayed or suppressed by use of a DNA construct comprising a silencing nucleic acid sequence which is effective for significantly reducing or eliminating the expression of MaMADS1 or MaMADS2 or both in the fruit. The silencing nucleic acid sequence in this construct is operatively linked to a promoter effective for expression in the fruit. The fruit of plants transformed with this construct exhibit significantly delayed ripening in comparison to fruit from non-transformed plants.

### CROSS-REFERENCE TO RELATED APPLICATIONS

This application claims the benefit under 35 U.S.C. 1.19(e) of U.S. provisional 61/515,351 filed Aug. 5, 2011, the contents of which are incorporated by reference herein.

### BACKGROUND OF THE INVENTION

1. Field of the Invention

The invention is drawn to transgenic banana plants wherein the ripening of the fruit is delayed.

2. Description of the Prior Art

Banana includes members of the genus Musa encompassing traditional dessert banana (e.g. Cavendish) and plantains that are important food staples in Asia, South/Central America and Africa. Banana fruit are susceptible to postharvest loss due to rapid ripening and associated short shelf life. Considerable effort and expense is spent on ripening control in banana to allow shipment from producing countries (Central/South America, Asia, Africa) to export markets such as the US, Japan and Europe or to local markets of these same countries. Approximately 20% of world banana production is exported at a value of over $5 billion annually. The remainder is consumed locally and represents a staple for over 400 million people in mostly developing countries. The current state-of-the-art in banana ripening control for export involves early harvest, environmental control to minimize ethylene exposure and eventual ethylene treatment to promote ripening. Expensive transport, storage and treatment facilities are required to manage this production system and large amounts of fruit are lost that would otherwise be available for consumption in developing countries where most bananas and plantains are grown. While resistance to transgenic crops (and especially those consumed as fresh products) is high in many countries, it is noteworthy that virtually no breeding is done in banana meaning that transgenic approaches are the only current method for targeted genetic modification. Critical disease problems that may challenge the ability to engage in future commercial banana production have resulted in much transgenic research on banana for managing pathogens.

### SUMMARY OF THE INVENTION

We have discovered that the ripening of banana fruit may be delayed or suppressed by the usage of a DNA construct comprising a silencing nucleic acid sequence which is effective for significantly reducing or eliminating the expression of MaMADS1 or MaMADS2 or both in the fruit. The silencing nucleic acid sequence in this construct is operatively linked to a promoter effective for expression in the fruit. The fruit of plants transformed with this construct exhibit significantly delayed ripening in comparison to fruit from non-transformed plants.

The transgenic plants of this invention which comprise fruit exhibiting significantly delayed ripening may be produced from any banana plant, tissue or cell which is capable of regeneration, by transformation with the construct. Transformed plants, plant tissue or plant cells comprising the construct are selected, and the transgenic plant is regenerated therefrom.

In accordance with this discovery, it is an object of this invention to provide a method for producing banana plants which produce fruit exhibiting delayed ripening.

It is another object of this invention to provide banana plants which produce fruit exhibiting delayed ripening.

A further object of this invention is to provide banana fruit having an extended shelf-life after harvest.

Other objects and advantages of this invention will become readily apparent from the ensuing description.

### BRIEF DESCRIPTION OF THE DRAWINGS

FIG. 1 shows the determination of MaMADS1 and MaMADS2 expression levels in their corresponding knock-down transgenic plants. MaMADS1 expression (A) was determined in two MaMADS1 RNAi transgenic plants and MaMADS2 expression (B, C) was determined in RNAi transgenic plants of MaMADS2 (B) and in transgenic plants of antisense MaMADS2 (C). The expression was determined in peel and pulp of fruit at breaker stage. Banana fruits of first hand were used in A and of the third hand in B and C. Sampling times (DAH) were: for control fruit in A-12 d (con 1) and 14 d (con 2), and in B and C-10 d; for MaMADS1 RNAi (A)—19 d (plant 19) and 23 d (plant 20); for MaMADS2 RNAi (B)—16 d (plants 21,23,24) and for antisense MaMADS2 (C)—19 d (plant 36), 16 d (plant 37) and 24 d plants 40,45). Primers used for the expression analysis are described in Supplemental Table, S3.

FIG. 2 shows a comparison of ethylene and CO₂ production after harvest between control and knock-down MaMADS1 and MaMADS2 fruits. Production was determined for control fruits (A, C) at two independent experiments; Control I on May (A) and Control II on June (C). Control I fruit served as control for transgenic RNAi MaMADS1 (B) and Control II for either RNAi MaMADS2 (D) or antisense MaMADS2 (E). Measurement for control I and RNAi MaMADS1 banana was performed on fruit of the third hand and that for Control II and RNAi MaMADS2 (D) or antisense MaMADS2 (E) on fruit the first hand.

FIG. 3 shows the quality parameters of knock-down MaMADS1 and MaMADS2 banana fruit. The parameters of color)(h°, firmness (N), and TSS (Brix) were examined in peel and pulp after harvest in MaMADS1 RNAi third hand (A) and MaMADS2 RNAi (B)/antisense (C) first hand and in the corresponding control plants. Summary of parameters is described in Table 5.

FIG. 4 shows the determination of expression levels of MaMADS2 in MaMADS1 knockdown and MaMADS1 in MaMADS2 knockdown transgenic plants. MaMADS2 expression (A) was determined in two MaMADS1 RNAi transgenic plants and MaMADS1 expression (B, C) was determined in RNAi transgenic plants of MaMADS2 (B) and in transgenic of antisense MaMADS2 (C). Obtaining of samples for analysis is described in FIG. 1. Primers used for the expression analysis are described in Table 4.

FIG. 5 shows the expression patterns of MaMADS3, MaMADS4 and MaMADS5 in knock-down MaMADS1 and MaMADS2 fruits. Expression was determined in MaMADS1 RNAi and MaMADS2 RNAi/antisense transgenic plants at samples described in FIG. 1. Primers used for the expression analysis are described in Table 4.

FIG. 6 shows the description of the gene segments used for constructing of the three types of the vectors which were used for banana transformation described in Table 1. Both MaMADS1 and MaMADS2 partial sequences are presented (each including sections of the K, C or 3′UTR regions). Vertical line indicates the border between the K and the C regions. The shading shows the sequence used for MaMADS1 pK+C RNAi (position 333-528 nucleotides) and MaMADS2 C+3′UTR RNAi and antisense (position 520-822). The letters pK denotes a partial sequence of the K region.

FIG. 7 shows the verification of inserts in the various transgenic lines. Schematic presentation of the pHELLSGATE and in the pBIN vectors are depicted in A and B, respectively. Schemes in A (reactions a and b) describe the anticipated PCR products from inserts made in pHELLSGATE vectors, while in B (reaction c) describes that made in pBin vector. The primers yielding these products are listed in Table 3.

FIG. 8 shows the ripening parameter of control and knock-down MaMADS1 and MaMADS2 banana fruits following ethylene treatment. Control and knockdown of MaMADS1 and MaMADS2 banana fruit were treated with ethylene (10 μl/L for A or 1 μl/L for B and C) immediately after harvest for one day and firmness or color was recorded in treated fruit 2 and 5 days after exposure.

### DEFINITIONS

The following terms are employed herein:

Cloning. The selection and propagation of (a) genetic material from a single individual, (b) a vector containing one gene or gene fragment, or (c) a single organism containing one such gene or gene fragment.

Cloning Vector. A plasmid, virus, retrovirus, bacteriophage, cosmid, artificial chromosome (bacterial or yeast), or nucleic acid sequence which is able to replicate in a host cell, characterized by one or a small number of restriction endonuclease recognition sites at which the sequence may be cut in a predetermined fashion, and which may contain an optional marker suitable for use in the identification of transformed cells, e.g., tetracycline resistance or ampicillin resistance. A cloning vector may or may not possess the features necessary for it to operate as an expression vector.

Codon. A DNA sequence of three nucleotides (a triplet) which codes (through mRNA) for an amino acid, a translational start signal, or a translational termination signal. For example, the nucleotide triplets TTA, TTG, CTT, CTC, CTA, and CTG encode for the amino acid leucine, while TAG, TAA, and TGA are translational stop signals, and ATG is a translational start signal.

DNA Coding Sequence. A DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, procaryotic sequences and cDNA from eukaryotic mRNA. A polyadenylation signal and transcription termination sequence will usually be located 3′ to the coding sequence.

DNA Construct. Artificially constructed (i.e., non-naturally occurring) DNA molecules useful for introducing DNA into host cells, including chimeric genes, expression cassettes, and vectors.

DNA Sequence. A linear series of nucleotides connected one to the other by phosphodiester bonds between the 3′ and 5′ carbons of adjacent pentoses. Expression. The process undergone by a structural gene to produce a polypeptide. Expression requires transcription of DNA, post-transcriptional modification of the initial RNA transcript, and translation of RNA.

Expression Cassette. A chimeric nucleic acid construct, typically generated recombinantly or synthetically, which comprises a series of specified nucleic acid elements that permit transcription of a particular nucleic acid in a host cell. In an exemplary embodiment, an expression cassette comprises a heterologous nucleic acid to be transcribed, operably linked to a promoter. Typically, an expression cassette is part of an expression vector.

Expression Control Sequence. Expression control sequences are DNA sequences involved in any way in the control of transcription or translation and must include a promoter. Suitable expression control sequences and methods of making and using them are well known in the art.

Expression Vector. A nucleic acid which comprises an expression cassette and which is capable of replicating in a selected host cell or organism. An expression vector may be a plasmid, virus, retrovirus, bacteriophage, cosmid, artificial chromosome (bacterial or yeast), or nucleic acid sequence which is able to replicate in a host cell, characterized by a restriction endonuclease recognition site at which the sequence may be cut in a predetermined fashion for the insertion of a heterologous DNA sequence. An expression vector may include the promoter positioned upstream of the site at which the sequence is cut for the insertion of the heterologous DNA sequence, the recognition site being selected so that the promoter will be operatively associated with the heterologous DNA sequence. A heterologous DNA sequence is “operatively associated” with the promoter in a cell when RNA polymerase which binds the promoter sequence transcribes the coding sequence into mRNA which is then in turn translated into the protein encoded by the coding sequence.

Fusion Protein. A protein produced when two heterologous genes or fragments thereof coding for two different proteins not found fused together in nature are fused together in an expression vector. For the fusion protein to correspond to the separate proteins, the separate DNA sequences must be fused together in correct translational reading frame.

Gene. A segment of DNA which encodes a specific protein or polypeptide, or RNA.

Genome. The entire DNA of an organism. It includes, among other things, the structural genes encoding for the polypeptides of the substance, as well as operator, promoter and ribosome binding and interaction sequences.

Heterologous DNA. A DNA sequence inserted within or connected to another DNA sequence which codes for polypeptides not coded for in nature by the DNA sequence to which it is joined. Allelic variations or naturally occurring mutational events do not give rise to a heterologous DNA sequence as defined herein.

Hybridization. The pairing together or annealing of single stranded regions of nucleic acids to form double-stranded molecules.

Nucleotide. A monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. The base characterizes the nucleotide. The four DNA bases are adenine (“A”), guanine (“G”), cytosine (“C”), and thymine (“T”). The four RNA bases are A, G, C, and uracil (“U”).

Operably Linked, Encodes or Associated. Operably linked, operably encodes or operably associated each refer to the functional linkage between a promoter and nucleic acid sequence, wherein the promoter initiates transcription of RNA corresponding to the DNA sequence. A heterologous DNA sequence is “operatively associated” with the promoter in a cell when RNA polymerase which binds the promoter sequence transcribes the coding sequence into mRNA which is then in turn translated into the protein encoded by the coding sequence.

Phage or Bacteriophage. Bacterial virus many of which include DNA sequences encapsidated in a protein envelope or coat (“capsid”). In a unicellular organism a phage may be introduced by a process called transfection.

Plant. Plant refers to a unicellular organism or a multicellular differentiated organism capable of photosynthesis, including algae, angiosperms (monocots and dicots), gymnosperms (ginko, cycads, gnetophytes, and conifers), bryophytes, ferns and fern allies. Plant parts are parts of multicellular differentiated plants and include seeds, pollen, embryos, flowers, fruits, shoots, leaves, roots, stems, explants, etc.

Plant Cell. Plant cell refers to the structural and physiological unit of multicellular plants. Thus, the term plant cell refers to any cell that is a plant or is part of, or derived from, a plant. Some examples of cells encompassed by the present invention include differentiated cells that are part of a living plant, differentiated cells in culture, undifferentiated cells in culture, and the cells of undifferentiated tissue such as callus or tumors.

Plasmid. A non-chromosomal double-stranded DNA sequence comprising an intact “replicon” such that the plasmid is replicated in a host cell. When the plasmid is placed within a unicellular organism, the characteristics of that organism may be changed or transformed as a result of the DNA of the plasmid. A cell transformed by a plasmid is called a “transformant.”

Polypeptide. A linear series of amino acids connected one to the other by peptide bonds between the alpha-amino and carboxy groups of adjacent amino acids.

Promoter. A DNA sequence within a larger DNA sequence defining a site to which RNA polymerase may bind and initiate transcription. A promoter may include optional distal enhancer or repressor elements. The promoter may be either homologous, i.e., occurring naturally to direct the expression of the desired nucleic acid, or heterologous, i.e., occurring naturally to direct the expression of a nucleic acid derived from a gene other than the desired nucleic acid. A promoter may be constitutive or inducible. A constitutive promoter is a promoter that is active under most environmental and developmental conditions. An inducible promoter is a promoter that is active under environmental or developmental regulation, e.g., upregulation in response to wounding of plant tissues. Promoters may be derived in their entirety from a native gene, may comprise a segment or fragment of a native gene, or may be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. It is further understood that the same promoter may be differentially expressed in different tissues and/or differentially expressed under different conditions.

Reading Frame. The grouping of codons during translation of mRNA into amino acid sequences. During translation the proper reading frame must be maintained. For example, the DNA sequence may be translated via mRNA into three reading frames, each of which affords a different amino acid sequence.

Recombinant DNA Molecule. A hybrid DNA sequence comprising at least two DNA sequences, the first sequence not normally being found together in nature with the second.

Ribosomal Binding Site. A nucleotide sequence of mRNA, coded for by a DNA sequence, to which ribosomes bind so that translation may be initiated. A ribosomal binding site is required for efficient translation to occur. The DNA sequence coding for a ribosomal binding site is positioned on a larger DNA sequence downstream of a promoter and upstream from a translational start sequence.

Replicon. Any genetic element (e.g., plasmid, chromosome, virus) that functions as an autonomous unit of DNA replication in vivo, i.e., capable of replication under its own control.

Start Codon. Also called the initiation codon, is the first mRNA triplet to be translated during protein or peptide synthesis and immediately precedes the structural gene being translated. The start codon is usually AUG, but may sometimes also be GUG.

Stringent Hybridization Conditions. The term “stringent conditions” or “stringent hybridization conditions” includes reference to conditions under which a probe will hybridize to its target sequence, to a detectably greater degree than to other sequences (e.g., at least 2-fold over background). Stringent conditions are sequence-dependent and will differ in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length. Typically, stringent hybridization conditions comprise hybridization in 50% formamide, 1 M NaCl, 1% SDS at 37° C., and a wash in 0.1×SSC at 60 to 65° C. It is also understood that due to the advances in DNA PCR and sequencing approaches that issues of gene identity and homology may be determined by sequence based rather than hybridization approaches.

Structural Gene. A DNA sequence which encodes through its template or messenger RNA (mRNA) a sequence of amino acids characteristic of a specific polypeptide.

Transform. To change in a heritable manner the characteristics of a host cell in response to DNA foreign to that cell. An exogenous DNA has been introduced inside the cell wall or protoplast. Exogenous DNA may or may not be integrated (covalently linked) to chromosomal DNA making up the genome of the cell. In prokaryotes and yeast, for example, the exogenous DNA may be maintained on an episomal element such as a plasmid. With respect to eucaryotic cells, a stably transformed cell is one in which the exogenous DNA has been integrated into a chromosome so that it is inherited by daughter cells through chromosome replication. This stability is demonstrated by the ability of the eucaryotic cell to establish cell lines or clones comprised of a population of daughter cells containing the exogenous DNA.

Transcription. The process of producing mRNA from a structural gene.

Transgenic plant. A plant comprising at least one heterologous nucleic acid sequence that was introduced into the plant, at some point in its lineage, by genetic engineering techniques. Typically, a transgenic plant is a plant that is transformed with an expression vector. It is understood that a transgenic plant encompasses a plant that is the progeny or descendant of a plant that is transformed with an expression vector and which progeny or descendant retains or comprises the expression vector. Thus, the term “transgenic plant” refers to plants which are the direct result of transformation with a heterologous nucleic acid or transgene, and the progeny and descendants of transformed plants which comprise the introduced heterologous nucleic acid or transgene.

Translation. The process of producing a polypeptide from mRNA.

### DETAILED DESCRIPTION OF THE INVENTION

This invention relates to the repression of two genes isolated from ripening banana fruit, termed MaMADS1 and MaMADS2. The genes are normally induced during banana ripening, and the invention utilizes gene specific sequences in DNA constructs to affect repression of the endogenous MaMADS1 or MaMADS2 genes in transgenic plants. We have determined that both are highly expressed specifically in the fruit. MaMADS2 acts in the pulp, and its expression precedes the increase in ethylene production. In contrast, MaMADSD1 is expressed in the pulp coincident with ethylene production, and with a greater increase in expression later during ripening. MaMADS1, and to a lesser extent MaMADS2, are expressed in the peel coincidentally with the increase in ethylene production. We have now cloned MaMADS1 and MaMADS2 from banana fruit cultivar Grand Nain, and these genes comprise the nucleotide sequences SEQ ID NO: 1 and SEQ ID NO: 2, respectively [Elitzur et al., 2010, J. Exp. Bot., 61(5):1523-1535, the contents of which is incorporated by reference herein]. The gene sequences for MaMADS1 and MaMADS2 have been deposited in GenBank as sequences EU869307 and EU869306, respectively, the contents of each of which are incorporated by reference herein. Each of the MaMADS1 and MaMADS2 genes include the translated regions or domains designated as MADS (or M), I, K and C, as well as untranslated regions. The M, I, K and C regions correspond to positions 0-228, 229-285, 286-528, and 529-705 of the MaMADS1 gene, and positions 0-228, 229-276, 277-519, and 520-732 of the MaMADS2 gene, respectively.

The process of the invention described herein may be used to produce banana fruit having delayed ripening in comparison to untreated or wild-type bananas (i.e., plants having fruit expressing the MaMADS1 and MaMADS2 genes at wild type levels). In accordance with this invention, a DNA construct comprising a silencing nucleic acid sequence which is effective for significantly inhibiting, reducing or eliminating the expression of the endogenous MaMADS1 or MaMADS2, is introduced and expressed in the plant. Expression of the silencing nucleic acid sequence reduces or eliminates the expression of either the MaMADS1 or MaMADS2 in the fruit, thereby delaying ripening in comparison to normal fruit. As used herein, a “silencing nucleic acid sequence” refers to a sequence that when transcribed results in the reduction of expression of one or more target genes, i.e., MaMADS1 and/or MaMADS2. A silencing nucleotide sequence may involve the use of RNA interference (RNAi) or antisense RNA, targeted to a single target gene, or the use of RNAi or antisense RNA, comprising two or more than two sequences that are linked or fused together and targeted to two or more than two target genes. The fused or linked sequences may be immediately adjacent to each other, or there may be linker fragment between the sequences. The “reduction of gene expression” or reduction of expression” refers to the reduction in the level of mRNA, protein, or both mRNA and protein, encoded by a gene or nucleotide sequence of interest. Reduction of gene expression may arise as a result of the lack of production of full length RNA, for example mRNA, or through cleaving the mRNA, or through inhibition of translation of the mRNA.

RNAi, as used herein, refers to the gene silencing mechanism involving small interfering RNA (siRNA) and microRNA (miRNA). In brief, RNAi techniques utilize the ability of double stranded RNA (dsRNA) to direct the degradation of mRNA sequences complementary to one of the strands. The RNAi mechanism can be initiated by transformation of the host plant with a silencing nucleic acid sequence that expresses a dsRNA, which dsRNA is processed by the natural DICER enzyme of the host cell to form siRNAs. The siRNAs then unwind into two single stranded RNAs (ssRNA), one of which functions as guide strand which incorporates into a RNA-degrading complex (RISC). Upon base pairing of the guide strand with a complementary mRNA molecule, the mRNA is cleaved by the RISC. The dsRNA expressed by the construct may comprise either intra- or intermolecular duplexes or hairpin configurations. Thus, in a preferred embodiment, the silencing nucleic acid sequence may comprise a pair of DNA sequences, one of which is complementary to all or a portion of the MaMADS1 and/or MaMADS2 gene sequences, and the other sequence comprising the same DNA sequence linked in its reverse orientation (i.e., the DNA sequences are in sense and antisense orientation). Upon transcription of the silencing nucleic acid, the single stranded mRNA transcribed from the first member of the pair will base-pair with the reverse oriented complementary strand transcribed from the second member of the pair to form dsRNA.

Silencing by antisense RNA utilizes nucleic acid molecules that are complementary to at least a portion of an mRNA of the MaMADS1 and/or MAMADS2 genes, whereby the antisense nucleic acid will hybridize to its corresponding mRNA, forming a double stranded molecule. This double stranded molecule has been shown to interfere with the transcription, stability (likely through mechanisms similar to those employed by RNAi) and/or translation of the mRNA.

For either of the RNAi or antisense techniques, the MaMADS1 or MaMADS2 gene that is targeted for inhibition or silencing within the plant may be inhibited or silenced using an isolated nucleic acid sequence encoding all or a portion of the MaMADS1 or MaMADS2 genes or their complements. Examples of sequences that may be used for silencing include a portion of any of the nucleotide sequence defined in SEQ ID NO:1 (MaMADS1) or SEQ ID NO:2 (MaMADS2), a nucleotide sequence that exhibits from about 80 to about 100% sequence identity to the nucleotide sequence defined in SEQ ID NO:1 or SEQ ID NO:2, or a nucleotide sequence that hybridizes to the nucleotide sequence defined in SEQ ID NO:1 or its complement, or to the nucleotide sequence defined in SEQ ID NO:2 or its complement, under stringent hybridization conditions, as defined above. In a preferred embodiment, the sequences used for silencing comprise contiguous nucleotides of the MaMADS1 or MaMADS2 genes, or their complements, in sense and/or antisense orientation. Although it is envisioned that any portion of the MaMADS1 or MaMADS2 genes may be used for silencing, in a preferred embodiment those portions which are not highly conserved among other MADS genes are preferred. Thus, preferred sequences that are used for silencing include those from the I or K domains of MaMADS1 or MaMADS2, and most preferably from the C domain and untranslated regions of MaMADS1 or MaMADS2. Conversely, although operable, portions exclusively from the M domain of the genes are not preferred as this region is highly conserved among the MADS genes of which there are many 10s to well over 100 in most characterized plant genomes. The length of the sequences that may be used for the silencing nucleic acid is not critical and may vary somewhat in accordance with the particular silencing technique used, i.e., either antisense or RNAi, but will typically vary between about 75 to 1,000 nucleotides. However, because double stranded RNA is typically cleaved by the Dicer enzyme in the RNAi pathway of the host cell into short fragments of approximately 15 to 30 nucleotides, it is envisioned that the length of the sequences may be less than 75 nucleotides, and as small as 30 nucleotides. For antisense applications, sequences of about 15 nucleotides may be used, although improved gene reduction of gene expression may be achieved using longer sequences.

The silencing nucleic acid sequence in this construct is operatively linked to a promoter which is active (i.e., functional or effective for expression) in the banana fruit. The promoter should provide a level of expression that the fruit of plants transformed with this construct will exhibit significantly delayed ripening in comparison to fruit from non-transformed plants. A variety of promoters are effective for use herein, and include both constitutive and inducible promoters. Without being limited thereto, the preferred promoter for use herein is the constitutive ³⁵S RNA promoter of CaMV (Odell et al. 1985, Nature, 313:810-812). By way of example, other suitable promoters which may be used include: the full-length transcript promoter from Figwort Mosaic Virus (FMV) (Gowda et al., 1989, J. Cell Biochem., 13D:301); the coat protein promoter to TMV (Takamatsu et al., 1987, EMBO J., 6:307); the light-inducible promoter from the small subunit of ribulose bis-phosphate carboxylase (ssRUBISCO) (Coruzzi et al, 1984, EMBO J. 3:1671; and Broglie et al., 1984, Science 224:838); mannopine synthase promoter (Velten et al., 1984, EMBO J., 3:2723); nopaline synthase (NOS) and octopine synthase (OCS) promoters (carried on tumor-inducing plasmids of Agrobacterium tumefaciens); and heat shock promoters, e.g., soybean hsp17.5-E or hsp17.3-B (Gurley et al., 1986, Mol. Cell. Biol., 6:559; and Severin et al., 1990, Plant Mol. Biol., 15:827). Other inducible promoters include those induced by chemical means, such as the yeast metallothionein promoter which is activated by copper ions (Mett et al., 1983, Proc. Natl. Acad. Sci., U.S.A. 90:4567); In2-1 and In2-2 regulator sequences which are activated by substituted benzenesulfonamides, e.g., herbicide safeners (Hershey et al., 1991, Plant Mol. Biol., 17:679); and the GRE regulatory sequences which are induced by glucocorticoids (Schena et al., 1991, Proc. Natl. Acad. Sci., U.S.A. 88:10421). While the expression of the MaMADS1 and MaMADS2 genes in addition to the results of this invention indicate that the effects of these genes is restricted to the fruit, one could further insure the effects of this invention to fruit and specifically maturing/ripening tissues by using a fruit-specific promoter. Examples include the tomato E8 (Giovannoni et al., 1989, The Plant Cell, 1:53-63), polygalacturonase or PG (Nicholass et al., 1995, Plant Mol. Biol., 28:423-435) and 2A11 (Van Harren et al., 1991, Plant Mol. Biol., 17:615-630) promoters.

Various methods may be used to produce the DNA construct, expression cassette or vector comprising the silencing nucleic acid sequence and promoter for transformation of the desired banana plant or its tissue or cells. The skilled artisan is well aware of the genetic elements that must be present on an expression construct/vector in order to successfully transform, select and propagate the expression construct in host cells. Techniques for manipulation of nucleic acids encoding promoter and the silencing sequences such as subcloning nucleic acid sequences into expression vectors, labeling probes, DNA hybridization, and the like are described generally in Sambrook et al., [Molecular Cloning—A Laboratory Manual (2nd Ed.), Vol. 1-3, Cold Spring Harbor Laboratory, Cold Spring Harbor, N.Y., 1989] and Kriegler [Gene Transfer and Expression: A Laboratory Manual, 1990] or on public sites

DNA constructs comprising the promoter operably linked to the silencing nucleic acid's DNA sequence can be inserted into a variety of vectors. Typically, the vector chosen is an expression vector that is useful in the transformation of plants and/or plant cells. Moreover, the expression constructs will typically comprise restriction endonuclease sites to facilitate vector construction and ensure that the promoter is upstream of and in-frame with silencing nucleic acid sequence. Exemplary restriction endonuclease recognition sites include, but are not limited to recognition site for the restriction endonucleases NotI, AatII, SacII, PmeI HindIII, PstI, EcoRI, and BamHI.

The expression vector may be a plasmid, virus, cosmid, artificial chromosome, nucleic acid fragment, or the like. Such vectors can be constructed by the use of recombinant DNA techniques well known to those of skill in the art. The expression vector comprising the promoter sequence may then be transfected/transformed into the target host cells. Successfully transformed cells are then selected based on the presence of a suitable marker gene as disclosed below.

A variety of vectors may be used to create the expression constructs comprising silencing nucleic acid sequence and promoter. Numerous recombinant vectors are known and available to those of skill in the art and are suitable for use herein for the stable transfection of plant cells or for the establishment of transgenic plants [see e.g., Weissbach and Weissbach, 1989, Methods for Plant Molecular Biology, Academic Press; Gelvin et al., 1990, Plant Molecular Biology Manual: Genetic Engineering of plants, an Agricultural Perspective, A. Cashmore, Ed., Plenum: NY, 1983; pp 29-38; Coruzzi et al., 1983, The Journal of Biological Chemistry, 258:1399; and Dunsmuir et al., 1983, Journal of Molecular and Applied Genetics, 2:285; Sagi L, Panis B, S. R, H. S, De Smet C, Swennen R, and Cammue P A. Genetic transformation of banana and plantain (Musa spp.) via particle bombardment. Biotechnology 13: 481-485, 1995; Khanna H, Becker D, Kleidon J, and Dale J. Centrifugation assisted Agrobacterium tumefaciens-mediated transformation (CAAT) of embryogenic cell suspensions of banana (Musa spp. Cavendish AAA and Lady finger AAB). Molecular Breeding 14: 239-252, 2004; and Santos E, Remy S, Thiry E, Windelinckx S, Swennen R, and Sagi L. Characterization and isolation of a T-DNA tagged banana promoter active during in vitro culture and low temperature stress. BMC Plant Biology 9: 77, 2009]. The choice of the vector is influenced by the method that will be used to transform host plants, and appropriate vectors are readily chosen by one of skill in the art.

Typically, the plant transformation vectors will include the promoter sequences operably linked to silencing nucleic acid sequence (DNA sequence) in the sense and/or antisense orientation, and a selectable marker. Such plant transformation vectors may also include a transcription initiation start site, a ribosome binding site, an RNA processing signal, a transcription termination site, and/or a polyadenylation signal. The plant transformation vectors may also include additional regulatory sequences from the 3′-untranslated region of plant genes, e.g., a 3′ terminator region to increase mRNA stability of the mRNA, such as the PI-II terminator region of potato or the octopine or nopaline synthase (NOS) 3′ terminator regions. The expression constructs may further comprise an enhancer sequence. As is known in the art, enhancers are typically found 5′ to the start of transcription, they can often be inserted in the forward or reverse orientation, either 5′ or 3′ to the silencing sequence. Expression constructs prepared as disclosed herein may also include a sequence that acts as a signal to terminate transcription and allow for the poly-adenylation of the mRNA produced by the silencing sequences operably linked to the promoter. Termination sequences are typically located in the 3′ flanking sequence of the silencing sequences, which will typically comprise the proper signals for transcription termination and polyadenylation. Thus, in one embodiment, termination sequences are ligated into the expression vector 3′ of the silencing sequences to provide polyadenylation and termination of the mRNA. Terminator sequences and methods for their identification and isolation are known to those of skill in the art, see e.g., Albrechtsen et al., 1991, Nucleic Acids Res. April 25; 19(8):1845-1852, and WO/2006/013072. The transcription termination sequences comprising the expression constructs, may also be associated with known genes from the host organism. Yet other DNA sequences encoding additional functions may also be present in the vector, as is known in the art. For instance, in the case of Agrobacterium transformations, T-DNA sequences will also be included for subsequent transfer to plant chromosomes.

As noted above, plant transformation vectors typically include a selectable and/or screenable marker gene to allow for the ready identification of transformants. As is known in the art, marker genes are genes that impart a distinct phenotype to cells expressing the marker gene, such that transformed cells can be distinguished and/or selected from cells that do not have the marker (and thus have not incorporated the vector). Exemplary selectable marker genes include, but are not limited to, those encoding antibiotic resistance (e.g. resistance to hygromycin, kanamycin, bleomycin, G418, streptomycin or spectinomycin) and herbicide resistance genes (e.g., phosphinothricin acetyltransferase). In this embodiment, the marker genes encode a selectable marker which one can “select” for by chemical means, e.g., through the use of a selective agent (e.g., a herbicide, antibiotic, or the like). Alternatively, the marker genes may encode a screenable marker which is identified through observation or testing, e.g., by “screening”. Exemplary screenable markers include e.g., green fluorescent protein.

A variety of selectable marker genes are known in the art and are suitable for use herein. Some exemplary selectable markers are disclosed in e.g., Potrykus et al. (1985, Mol. Gen. Genet., 199:183-188); Stalker et al. (1988, Science, 242:419 422); Thillet et al. (1988, J. Biol. Chem., 263:12500 12508); Thompson et al. (1987, EMBO J. 6:2519-2523); Deblock et al. (1987, EMBO J. 6:2513-2518); U.S. Pat. No. 5,646,024; U.S. Pat. No. 5,561,236; U.S. Patent application Publication 20030097687; and Boutsalis and Powles (1995, Weed Research 35: 149-155).

Screenable markers suitable for use herein include, but are not limited to, a β-glucuronidase (GUS) or uidA gene, (see e.g., U.S. Pat. No. 5,268,463, U.S. Pat. No. 5,432,081 and U.S. Pat. No. 5,599,670); a β-gene (see e.g., Sutcliffe, 1978, Proc. Natl. Acad. Sci. USA, 75:3737-3741); β-galactosidase; and luciferase (lux) gene [see e.g., Ow et al., 1986, Science, 234:856-859; Sheen et al., 1995, Plant J., 8(5):777-784; and WO 97/41228]. Other suitable selectable or screenable marker genes also include genes which encode a “secretable marker” whose secretion can be detected as a means of identifying or selecting for transformed cells. Such secretable markers include, but are not limited to, secretable antigens that can be identified by antibody interaction (e.g., small, diffusible proteins detectable for example by ELISA); secretable enzymes which can be detected by their catalytic activity, such as small active enzymes detectable in extracellular solution (e.g., α-amylase, β-lactamase or phosphinothricin acetyltransferase); and proteins that are inserted or trapped in the cell wall (e.g., proteins that include a leader sequence such as that found in the expression unit of extensin or tobacco PR-S).

The DNA constructs containing the active promoter operably linked to the silencing DNA sequence can be used to transform banana embryonic culture tissue as described in the Example herein below, and thereby generate transgenic banana plants which produce fruit exhibiting significantly decreased expression of the MaMADS1 and/or MaMADS2 genes, and consequently delayed ripening. Embryonic cell suspension is produced from male flower as described by Strosse et al. (2003. Banana and plaintain embryogenic cell suspensions [Vezina and Picq, eds.]. INIBAP Technical Guidelines 8. The International Network for the Improvement of Banana and Plaintain, Montpellier, France. INIBAP ISBN: 2-910810-63-1, the contents of which is incorporated by reference herein; also at http://bananas.bioversityinternational.org/files/files/pdf/publications/tg8_en.pdf). Banana plants which may be transformed in accordance with this invention include any species of the genus Musa, including but not limited to M. acuminate (or M. acuminata), M. balbisiana, and M. acuminate×M. balbisiana. Particularly preferred cultivars are those which may be transformed including dessert and cooking banana.

Transformation of embryonic culture with the DNA construct comprising the silencing nucleic acid sequence operatively linked to the promoter may be affected using a variety of known techniques. Techniques for the transformation and regeneration of plant cells are well known in the art, see e.g., Weising et al., 1988, Ann. Rev. Genet. 22:421-477; U.S. Pat. No. 5,679,558; Khanna H, Becker D, Kleidon J, and Dale J. Centrifugation assisted Agrobacterium tumefaciens-mediated transformation (CAAT) of embryogenic cell suspensions of banana (Musa spp. Cavendish AAA and Lady finger AAB). Molecular Breeding 14: 239-252, 2004; and Santos E, Remy S, Thiry E, Windelinckx S, Swennen R, and Sagi L. Characterization and isolation of a T-DNA tagged banana promoter active during in vitro culture and low temperature stress. BMC Plant Biology 9: 77, 2009. A variety of techniques are suitable for use herein, and include, but are not limited to, electroporation, microinjection, microprojectile bombardment, also known as particle acceleration or biolistic bombardment, viral-mediated transformation, and Agrobacterium-mediated transformation. Detailed descriptions of transformation/transfection methods are disclosed, for example, as follows: direct uptake of foreign DNA constructs (see e.g., EP 295959); techniques of electroporation [see e.g., Fromm et al., 1986, Nature (London) 319:791]; high-velocity ballistic bombardment with metal particles coated with the nucleic acid constructs [see e.g., Kline et al., 1987, Nature (London) 327:70, and U.S. Pat. No. 4,945,050]; methods to transform foreign genes into commercially important crops, such as rapeseed [see De Block et al., 1989, Plant Physiol. 91:694-701], sunflower [Everett et al., 1987, Bio/Technology 5:1201], soybean [McCabe et al., 1988, Bio/Technology 6:923; Hinchee et al., 1988, Bio/Technology 6:915; Chee et al., 1989, Plant Physiol. 91:1212 1218; Christou et al., 1989, Proc. Natl. Acad. Sci. USA 86:7500 7504; EP 301749], rice [Hiei et al., 1994, Plant J. 6:271 282], corn [Gordon-Kamm et al., 1990, Plant Cell 2:603-618; Fromm et al., 1990, Biotechnology 8:833 839], and Hevea (Yeang et al., In, Engineering Crop Plants for Industrial End Uses. Shewry, P. R., Napier, J. A., David, P. J., Eds. Portland: London, 1998, pp 55-64). Other suitable, known methods are disclosed in e.g., U.S. Pat. Nos. 5,597,945; 5,589,615; 5,750,871; 5,268,526; 5,262,316; and 5,569,831. In a preferred embodiment the transformation is effected using Agrobacterium-meditated transformation as described by Khanna H, Becker D, Kleidon J, and Dale [J. Centrifugation assisted Agrobacterium tumefaciens-mediated transformation (CAAT) of embryogenic cell suspensions of banana (Musa spp. Cavendish AAA and Lady finger AAB). Molecular Breeding 14: 239-252, 2004] and by Santos E, Remy S, Thiry E, Windelinckx S, Swennen R, and Sagi L. [Characterization and isolation of a T-DNA tagged banana promoter active during in vitro culture and low temperature stress. BMC Plant Biology 9: 77, 2009], the contents of each of which are incorporated by reference herein.

Agrobacterium tumefaciens-meditated transformation techniques are well described in the scientific literature. See, e.g., Horsch et al. Science, 1984, 233:496-498, and Fraley et al., 1983, Proc. Natl. Acad. Sci. USA 80:4803. Typically, a plant cell, an explant, a meristem, a seed or in the case of banana embryonic culture is infected with Agrobacterium tumefaciens transformed with the expression vector/construct which comprises the promoter and silencing DNA sequence. Under appropriate conditions known in the art, the transformed plant cells are grown to form shoots, roots, and develop further into plants. The nucleic acid segments can be introduced into appropriate plant cells, for example, by means of the Ti plasmid of Agrobacterium tumefaciens. The Ti plasmid is transmitted to plant cells upon infection by Agrobacterium tumefaciens, and is stably integrated into the plant genome (Horsch et al., 1984, Inheritance of Functional Foreign Genes in Plants, Science, 233:496-498; and Fraley et al., 1983, Proc. Nat'l. Acad. Sci. U.S.A. 80:4803).

After transformation of the embryonic culture, those plant cells transformed with the selected vector such that the construct is integrated therein can be cultivated in a culture medium under conditions effective to grow the plant or its cell or tissue. Successful transformants may be differentiated and selected from non-transformed plants or cells using a phenotypic marker. As described above, these phenotypic markers include, but are not limited to, antibiotic resistance, herbicide resistance or visual observation.

Transformed embryogenic cells which are derived by any of the above transformation techniques can be cultured to regenerate a whole banana plant which possesses the desired transformed genotype of decreased MaMADS1 and/or MaMADS2 expression, and the phenotype of delayed fruit ripening. Plant regeneration techniques are well known in the art. For example, plant regeneration from cultured protoplasts is described in Evans et al., Protoplasts Isolation and Culture, Handbook of Plant Cell Culture, pp. 124-176, MacMillan Publishing Company, New York, 1983; and Binding, Regeneration of Plants, Plant Protoplasts, pp. 21-73, CRC Press, Boca Raton, 1985, all of which are incorporated herein by reference. Regeneration can also be obtained from plant callus, explants, organs, or parts thereof. Such regeneration techniques are described generally in Klee et al. 1987, Ann. Rev. of Plant Phys. 38:467-486, the contents of which is also incorporated by reference herein, and for banana regeneration is described by Strosse et al. ibid.

Banana plants successfully transformed with the silencing nucleic acid constructs are subsequently screened at an early stage of development at the DNA level to contain the desired constructs and later on during development of full plants to select for those exhibiting the desired constructs. Banana fruit which show decreased expression of the MaMADS1 and/or MaMADS2 genes are examined for fruit quality parameters. As used herein, banana plants which express the silencing nucleic acid sequences at an effective or sufficient level therein, will exhibit significantly reduced expression of MaMADSA1 and/or MaMADS2 genes, in comparison to non-transformed or wild-type control plants. Reduced expression of MaMADS1 or MaADS2 may be evidenced by measurement of a decrease in the amount or level of transcription product (mRNA) of these genes in the fruit of the transformed plants, or as described below, by delayed ripening of the fruit of these plants, all in comparison to the control plants. In a preferred embodiment, expression of the MaMADS1 and MaMADS2 genes mRNA transcription product is determined by quantitative RT-PCR. Alternatively, screening for the transformation events may be accomplished by Northern blot analysis of mRNA products [Kroczek, 1993, Chromatogr. Biomed. Appl., 618(1-2): 133-145]. The actual decrease in MaMADS1 or MaMADS2 gene expression will vary with the particular silencing nucleic acid sequences and promoter used, the maturity of the fruit at harvest, and the particular portion of the fruit analyzed (e.g., peel or pulp). The skilled artisan will also recognize that different independent transformation events will result in different levels and patterns of silencing (Jones et al., 1985, EMBO J., 4:2411 2418; and De Almeida et al., 1989, Mol. Gen. Genetics, 218:78 86), and thus that multiple events may need to be screened in order to obtain lines displaying the desired decrease in expression level of the MaMADS1 and/or MaMADS2 genes. However, transgenic banana plants produced in accordance with this invention will typically exhibit MaMADS1 and/or MaMADS2 transcription levels that are 50% (one half) or less, and preferably 35% or less, of those of a non-transformed control (measured at a confidence level of at least 80%, preferably measured at a confidence level of 95%).

In a preferred embodiment, the transformed plants are further screened for the desired production of fruit exhibiting delayed ripening after harvest. Fruit of transformed plants which express the silencing nucleic acid sequences at a sufficient level therein, will preferably exhibit significantly delayed ripening, in comparison to the fruit of non-transformed or wild-type control plants. As described in the Examples herein below, ripening time may be demonstrated, for example, by evaluation of fruit for peel color, firmness of the fruit flesh or determination of increased total soluble solids in the peel and/or pulp. Thus, delayed ripening may be evidenced by a significant increase in time for the harvested banana fruit from a transgenic plant harvested at a commercial stage (¾ of its final size) to achieve the same level of one or more of these ripening parameters, all in comparison to the untreated control. As with the decrease in MaMADS1 and MaMADS2 expression above, the actual delay in ripening exhibited by the resultant transgenic plants will vary with the silencing nucleic acid sequences and promoter used, as well as storage conditions of the harvested fruit. As a practical matter, transgenic banana plants produced in accordance with this invention will produce fruit exhibiting a delay in ripening of at least two days, preferably 3 days, and most preferably 10 days or more, all in comparison to a non-transformed control (measured at a confidence level of at least 80%, preferably measured at a confidence level of 95% when banana kept at 20° C.).

One of skill in the art will recognize that, after the construct comprising the silencing nucleic acid sequences operatively linked to a promoter is stably incorporated in transgenic plants and confirmed to be operable, plant tissue or plant parts of the transgenic plants may be harvested, and/or the seed collected in the case banana-producing seeds will be transformed. Edible banana cannot be propagated by seeds and therefore the trait will be transferred by propogation of new plantlets from the original transgenic plant or its propagules (propagants). Introduction of the trait to other banana species will be performed by new transformation.

The following examples are intended only to further illustrate the invention and are not intended to limit the scope of the invention which is defined by the claims.

### EXAMPLES

In this Example we examined the function of MaMADS1 and MaMADS2 by creating under-expressing (i.e. target gene repressed) transgenic banana plants. Ripening parameters of the banana fruit were determined in both transgenic and control plants. In addition, the response of the fruit to exogenous ethylene has been examined.

### Materials and Methods

### Plant Material

Banana (Musa acuminate, AAA Cavendish subgroup, Grand Nain) was used for transformation and the resulting plants were planted along the northern shore of Israel. Banana fruit of control and transgenic plants were harvested between May to June at three quarters of their final filling when they were not fully round in cross section. The maturity stage of the different fruit was verified by measuring the angles of the banana cross sections and the average of angles was similar in control and transgenic fruit (data not shown). Hands of the first, second or third tiers containing 10-30 fingers were separated from the bunch to monitor the green, as well as the climacteric and post-climacteric stages. Following separation, the cut area of the hands was sprayed with 0.1% thiobendazole to prevent crown rot decay, and the hands were air-dried, packed in polyethylene bags and stored at 20° C. and 95% RH. Samples were taken from pulp and peel separately on consecutive days, up to 35 days after harvest (DAH) and used for determination of ripening parameters, as well as for preparation of mRNA. When sensitivity to ethylene was determined, fruit were treated with ethylene at 1-10 μl L⁻¹, as indicated, for 20 h on the first to the third DAH.

### Determination of Ripening Parameters

Ethylene (C₂H₄) and carbon dioxide (CO₂) production were determined by sealing a banana finger in 2-L sealed glass jar at 20° C. as described (Elitzur et al., 2010, J Exp Bot. 61:1523-1535). Peel color was determined from surface area of three individual banana fingers using Minolta CR-300 (Minolta Corporation, New Jersey, USA). Firmness was measured in the middle of whole fruit using a Chatillon Force tester (Ametek Inc., Florida USA). Total soluble solids (TSS) were determined in the juice of peel and pulp resulting from freezing and thawing of the tissues, using a handheld HSR-500 refractometer (Atago Co. Ltd, Japan).

### Construction of Plasmids for Reduced Expression of MaMADS1 and MaMADS2 and Verification of Insertion

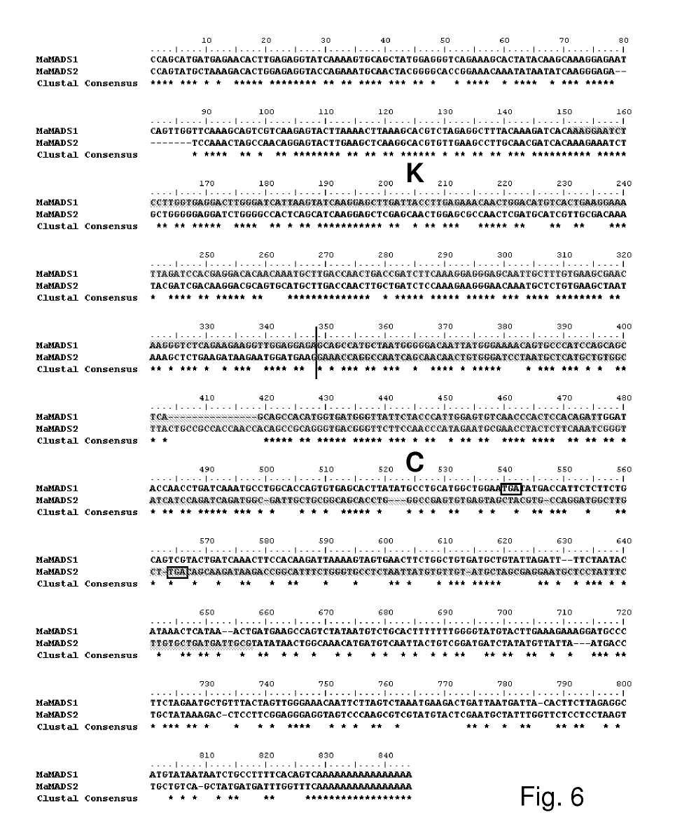

Three types of banana transgenic plants were created with reduced levels of either MaMADS1 or MaMADS2 (Table 1). The constructs included different sections of the genes which are described in FIG. 6. All constructs were under the control of the constitutive 35S promoter. An antisense construct of MaMADS2 was created by cloning a section of 303 by MaMADS2 C and 3′ UTR regions (FIG. 6), in a reverse orientation into a pBIN binary vector. The plasmid was generated by linker insertion at the NotI restriction site of the cloning multi site area creating XhoI and EcoRI sites. The plasmid contains the NPTII gene under the direction of the NOS promoter for Kanamycin selection. Cloning was performed by creating a PCR product using forward (FW) and reverse (RV) primers containing the XhoI and EcoRI restriction enzymes sites, respectively (Table 2).

Gateway technology (Invitrogen Inc., Carlsbad, Calif.) was used for preparation of the RNAi constructs. MaMADS1 sequences were cloned into pHellsgate2 (Genbank AJ311874) and both sections of MaMADS2 (C+3′UTR and K regions) into pHellsgate8 (Genbank AF489904, kindly provided by CSIRO Plant Industry, Can berra, Australia).

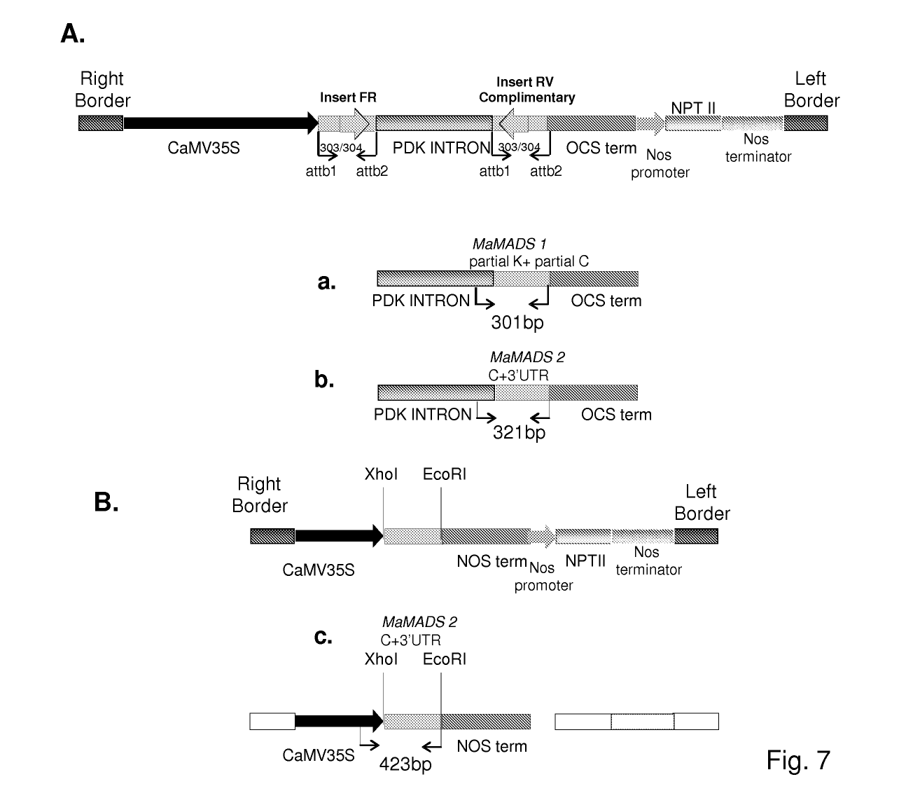

MaMADS1 and MaMADS2 target sequence regions were amplified from banana cDNA by PCR using the primers described in Table 2. The forward and the reverse primers for each of the sequences include the attB1 and the attB2 recombination sites. The corresponding PCR products were purified from the gel using the QIAquick PCR purification kit (Qiagen, Maryland, MD, USA) and cloned into vector pDONR 221 (Invitrogen Carlsbad, Calif., USA Cat. No. 12536-017), using Gateway BP Clonase II Enzyme Mix (Invitrogen Cat. No. 11789-020) mediated by the attB sites of the pDONR. A second Clonase step using LR Clonase II Enzyme Mix (Invitrogen Cat. No. 11791-020) mediated by the attL sites created on the pDONR and the attP or the attR sites of the entry vector which exist in pHellsgate2 and pHellsgate8, respectively (FIG. 7A). The plasmids were verified by restriction enzymes digest, PCR reactions and sequencing of PCR products from each plasmid (data not shown).

Following transformation the existence of the constructs was verified by PCR reactions on DNA preparations from leaves. DNA was prepared by using the Extract-N-Amp Plant PCR Kit (Sigma Aldrich XNAP2E). The reactions and the expected products are described in FIG. 7A and the primers locations used for these reactions are in FIG. 7. PCR products of few of the transgenic banana trees corresponding to the inserted constructs are depicted in FIG. 7B. The negative control fruit did not contain any of the plasmids; however they were developed as plants from the same embryonic culture.

### Transformation of Embryonic Banana Cultures

The constructs described above were used for banana transformation. The transformation of the banana was performed by RAHAN MERISTEM Ltd. Immature male flowers were used for the generation of embryonic callus (approximately 6 months). Once at hand, these calli were transferred to embryonic cell suspension (Schoofs, 1997, The origin of embryogenic cells in Musa., Leuven, Belgium). Cell clumps were used for transformation by Agrobacterium with the inclusion of kanamycin. The transformed somatic embryo were transferred to a medium containing half strength MS medium containing 10 μM zeatine for approximately six months until shoots were clearly visible. Following plantlet emergence, they were hardened in a greenhouse under mist until 4-leaflets stage. The detailed dates of handling the transgenic plants are described in Table 1.

Determination of Gene Expression by Quantitative RT-PCR (q-RT-PCR) Analysis

Total RNA was extracted and treated with TURBO DNase free (Applied Biosystems, USA) as described at the manufacturer manual. First strand DNA was prepared by Verso™ cDNA kit (Thermo Fisher Scientific Inc., USA), and used for q-RT-PCR analysis. Primers for this analysis were designed by Primer Express (v. 2; Applied Biosystems) and are described in Table 4 and their specificity for each of the genes has been demonstrated in a previous study (Elitzur et al., 2010, ibid). Primers concentrations was usually 4-8 μM and the cDNA usually was diluted 1:20 and 1:5000 for determination of the gene of interest and the reference Ribosomal gene, respectively. These concentrations were predetermined to enable linear and high efficient response. Reaction mixture contained cDNA, the appropriate forward and reverse primers and Power SYBR Green PCR Master mix (Applied Biosystems, USA) in a 20 μl total sample volume. Reactions were run in triplicates on a Rotor-Gene 3000 PCR machine (Corbett Life Research, Australia) using 35 cycles of 95° C. for 10 sec, 60° C. for 15 sec, and 72° C. for 20 sec.

The sequence of Glyceraldehyde-3-phosphate dehydrogenase (GAPDH) and ribosomal gene (AY821550 and EU433925, respectively) were used as reference for equalizing the levels of RNA. Forward and reverse primers for the references genes are: 5′-GCAAGGATGCCCCAATGT-3′ and 5′-AGCAAGACAGTTGGTTGTGCAG-3′ for GAPDH, 5′-GCGACGCATCATTCAAATTTC-3′ and 5′-TCCGGAATCGAACCCTAATTC-3′ for ribosomal gene.

Data obtained was analyzed with Rotor-Gene 6 software and the qBase quantification Software was used for calculations. The data is expressed according to the delta-delta-Ct method and results represent one experiment out of at least two independent sampling, for which usually two preparations of cDNA were examined.

### Results

Creation of Banana Transgenic Plants with Reduced Expression of Either MaMADS1 or MaMADS2

Our previous studies described the isolation of 6 full length MaMADS-box genes from banana and in this study we have examined the function of two genes: MaMADS1 and MaMADS2. Three types of constructs including different sections of the genes were created and used for banana transformation: a) RNAi MaMADS1 which include a 197 bp section of the K region and a 112 bp section of the C region; b) RNAi MaMADS2 which include a 212 bp section of the C region and a 91 bp region of the 3′UTR; c) Antisense MaMADS2 which include a the section used for b. The genes' sections described above are depicted in FIG. 6, and the creation of constructs and their verification are described in Materials and methods. The constructs were used for transformation of embryo cultures and the transgenic plants created are described in Table 1. The verification of insert has been performed as described in FIG. 7.

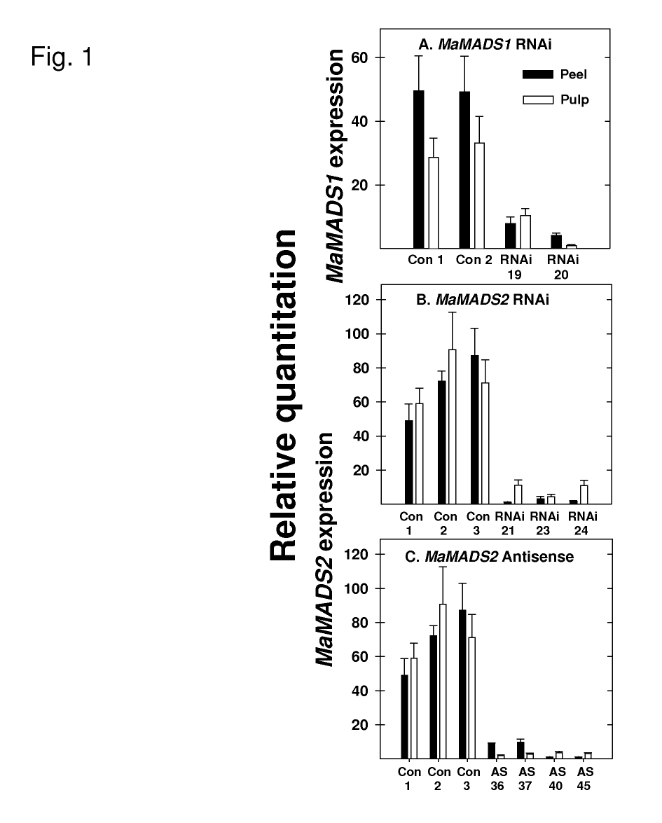

The transformation of the three constructs yielded fertile plants and banana fruit were harvested from independent positive trees of three types of the transgenic plants; RNAi MaMADS1, RNAi MaMADS2, and antisense MaMADS2, and from control plants. The transcript levels at breaker stage in peel and pulp of MaMADS1 were examined in RNAi MaMADS1 and that of MaMADS2 in RNAi MaMADS, as well as in antisense MaMADS2 (FIG. 1). The transcripts levels of the corresponding genes in control plants were high in peel and pulp, as expected; however the level of MaMADS1 was low in RNAi MaMADS1, and that of MaMADS2 was low in transgenic plants of RNAi MaMADS2, as well as in antisense MaMADS2 in peel and pulp in all plants examined. These results confirm that the transgenic plants are indeed reduced in the expression of either the MaMADS1 or the MaMADS2 genes.

### Ripening Characterization of MaMADS1 or MaMADS2 Knockdown Banana Fruit

Fruit for RNAi MaMADS1 and their corresponding control were harvested on May and those for RNAi MaMADS2 and antisense MaMADS2 and their control on June. It is clear that already following 19-20 days after harvest control banana fruit showed sever senescence symptoms, while banana fruit of most of the transgenic plants are at breaker or even at pre-breaker stage.

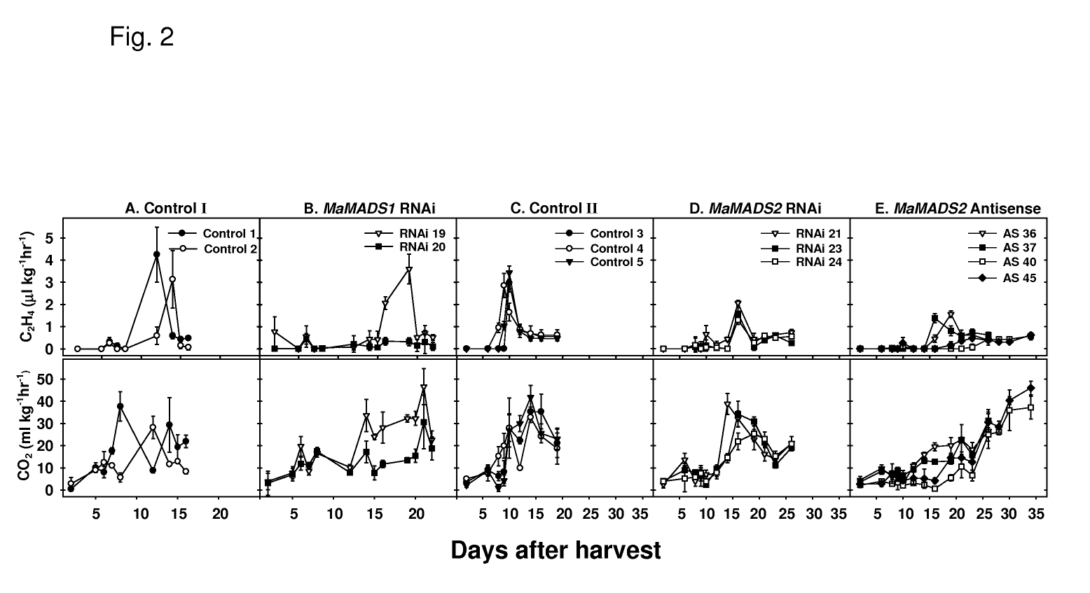

Respiration and ethylene production was measured on control and transgenic fruit on consecutive days from harvest until the appearance of brown dots (FIG. 2: Control I (A) for RNAi MaMADS1(B), and Control II (C) for RNAi MaMADS2 (D) as well as for antisense MaMADS2 (E)). In each of the control fruit in Control I group, the fruit respiration preceded the burst in ethylene production and climacteric respiration occurred between 9-12 DAH ethylene peak between 12-15 DAH (FIG. 2A). In comparison, fruit of two plants of RNAi MaMADS1 have been examined and in one (RNAi-20), no ethylene peak was detected, in parallel with only slight increment in respiration following 21 DAH. In fruit from the second tree (RNAi 19), respiration burst was observed on the 14th DAH which was followed by ethylene peak on the 19th DAH (FIG. 2B).

Among the control plants of Control II group the increase in ethylene production preceded the respiration peak and it occurred 10-11th DAH, while respiration peaked on the 15th DAH (FIG. 2C). In comparison, the ethylene peak in fruit from RNAi MaMADS2 transgenic plants was lower than in control fruit and appeared on the 16th DAH in parallel with increase in respiration (elevated respiration occurred between the 14-20th DAH in fruit from the three different plants) (FIG. 2D). Higher inhibition of ethylene production and respiration was observed in banana fruit from antisense MaMADS2 transgenic plants (FIG. 2E). In two plants (AS 40 and AS 45) the banana fruit did not produce any ethylene and in these fruits respiration incremented gradually starting from the 26th DAH. In two other plants the ethylene production peak was lower than that of control banana, and it occurred on the 16th (AS 37) or on the 19th (AS 36) DAH. The peaks in these two types of fruit occurred in parallel with increase in respiration. Note that only in two antisense MaMADS2 fruit which did not exhibit ethylene production, respiration incremented gradually, while in all other fruit respiration decreased following a high level.

The parameters of color, firmness and TSS were followed in the various fruit of control and transgenic plants and Table 5 describes the values of all these parameters in control and transgenic plants when fruit reached breaker stage. In general, in all fruits of transgenic plants, breaker was reached later than in controls and fruits of transgenic plant were of similar firmness and TSS. More specifically, changes in color, and increase in peel TSS occurred in parallel to the burst in ethylene production (on 12th and 15th DAH) in the two control plants of RNAi MaMADS1. However, changes in firmness and pulp TSS occurred at the same time in the two control plants (12th DAH). Fruit from two RNAi MaMADS1 transgenic plants exhibited delayed changes in all parameters which paralleled their reduced ethylene and respiration (FIG. 2B). While in fruit of one plant (RNAi 19), change in color and reduced respiration occurred on the 16th DAH (before the increase in ethylene production), in fruit from the other plant (RNAi 20), the change in color and firmness occurred on the 23rd DAH, and for the same color change (hue angle 100) the banana of RNAi MaMADS1 remained firmer. It should be noted, that the TSS of both peel and pulp of both RNAi MaMADS1 reached the same levels as of control, although at a stage of the same color (hue angle of 100), fruit of one plant (RNAi 19) had lower TSS levels in both peel and pulp, but in another (RNAi 20) TSS was similar to that of control in pulp but higher in peel. Another interesting phenomenon occurred in plant 20; while firmness and color change occurred in parallel, the TSS increased gradually before that.

The change in color, decrease in firmness and increase in TSS was similar in fruit from three independent plants of Control II group and it occurred concomitantly with the peak in ethylene production. In comparison, banana fruit from all three plants of RNAi MaMADS2, exhibited a similar delay in color break, a decrease in firmness and an increase in TSS. At mid color change (hue angle 100), banana of these transgenic plants were slightly firmer and had lower TSS in peel and pulp (Table 5).

Among the antisense MaMADS2 plants there were two distinct groups which exhibited slow rate of changes (AS 36, AS37) and very slow rate (AS 40, AS 45) and in comparison to that of control, in parallel with low rate or lack of ethylene production, respectively (FIG. 2E). At breaker stage all antisense MaMADS2 fruit were firmer than control; however, while the TSS of mainly the pulp of fruit from AS36 and A37 plants was lower than that of control, the TSS of fruit exhibiting the stronger delay (AS 40, AS 45) reached the same levels as that of control.

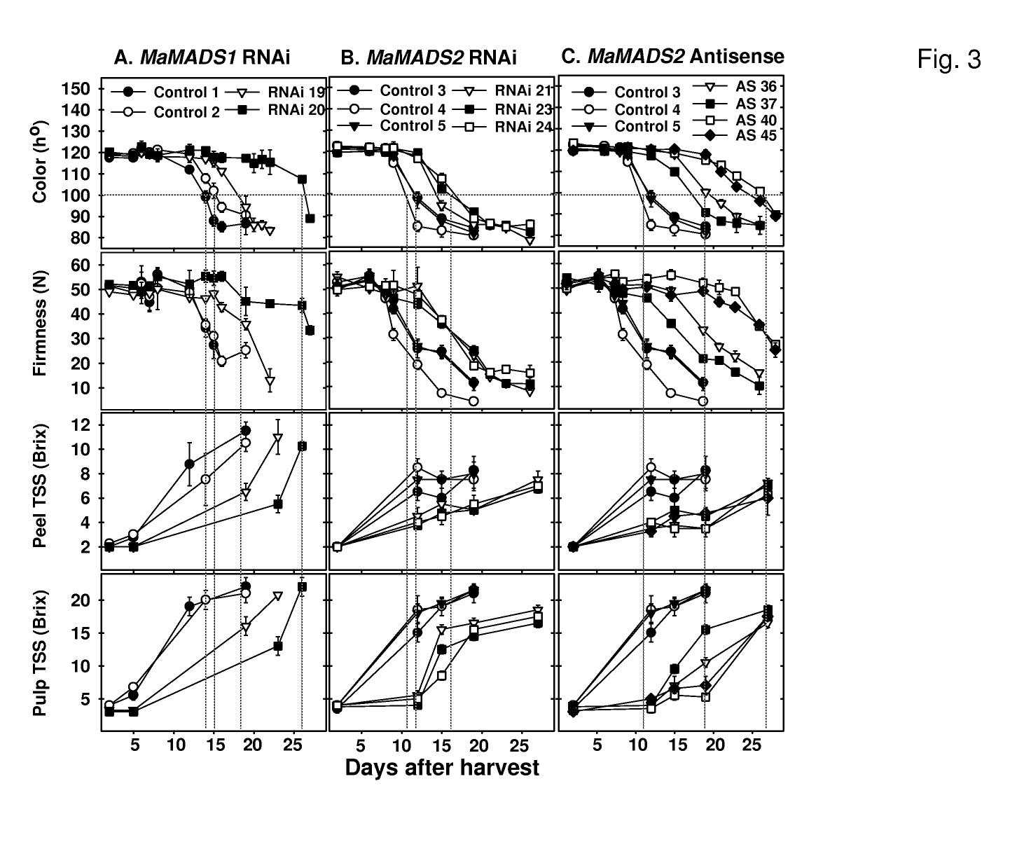

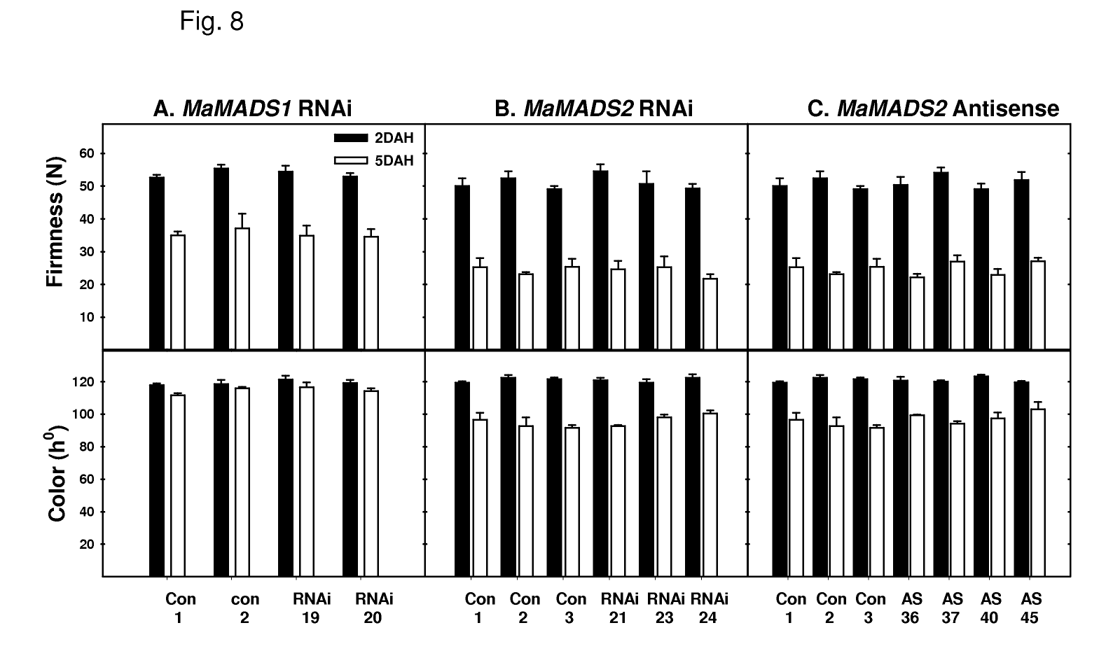

It was clear that normal ripening was delayed in fruit of the transgenic plants, and it was important to determine if these fruit exhibiting ripening delay following ethylene treatment. The banana fruit of the control and transgenic plants were exposed to ethylene (FIG. 3) and it can be seen that fruit of the transgenic plants responded to ethylene in a similar manner and developed yellow color. Examination of firmness and color showed that there was no difference between control and any of the transgenic plants. (FIG. 8).

### Analysis of MaMADS-Box Genes' Expression in the Fruit of MaMADS1 and MaMADS2 Downregulated Transgenic Plants

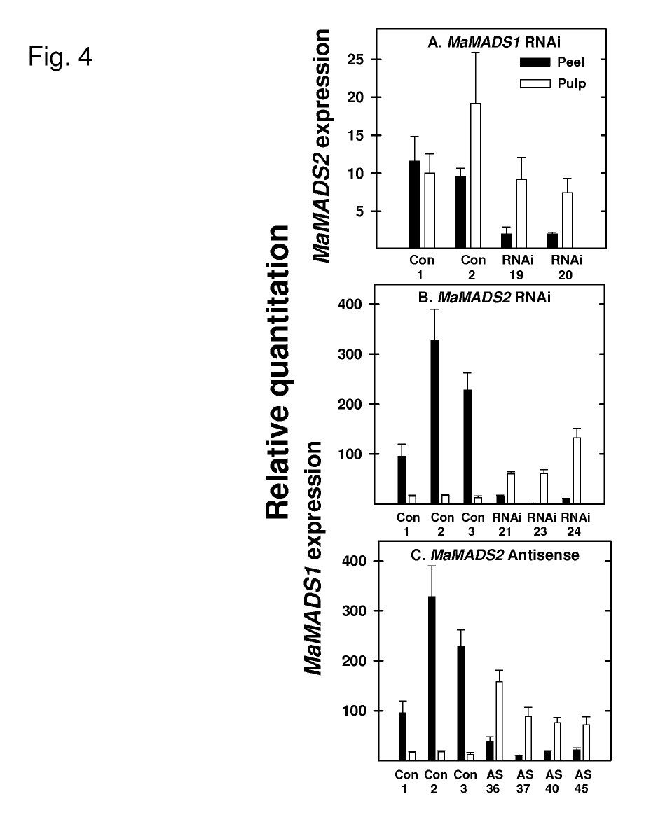

To elucidate the interactions between MaMADS1 and MaMADS2 genes' expression, we have determined the levels of expression of the reciprocal genes in the MaMADS1 and MaMADS2 downregulated transgenic plants (FIG. 4). The expression levels of MaMADS2 in control plants was similar between peel and pulp (FIG. 4A, and also FIG. 1B, C), and the levels of MaMADS1 was higher in peel than in pulp (FIG. 6B and also FIG. 1A). The down regulation of MaMADS1 did not reduce the levels of MaMADS2 in the pulp, but did reduce it in the peel (FIG. 4A). On the other hand, downregulation of MaMADS2 induced MaMADS1 in the pulp and reduced the levels of MaMADS1 in the peel, and similar pattern was observed in fruit obtained from RNAi MaMADS2 and antisense MaMADS2 (FIG. 4B, C).

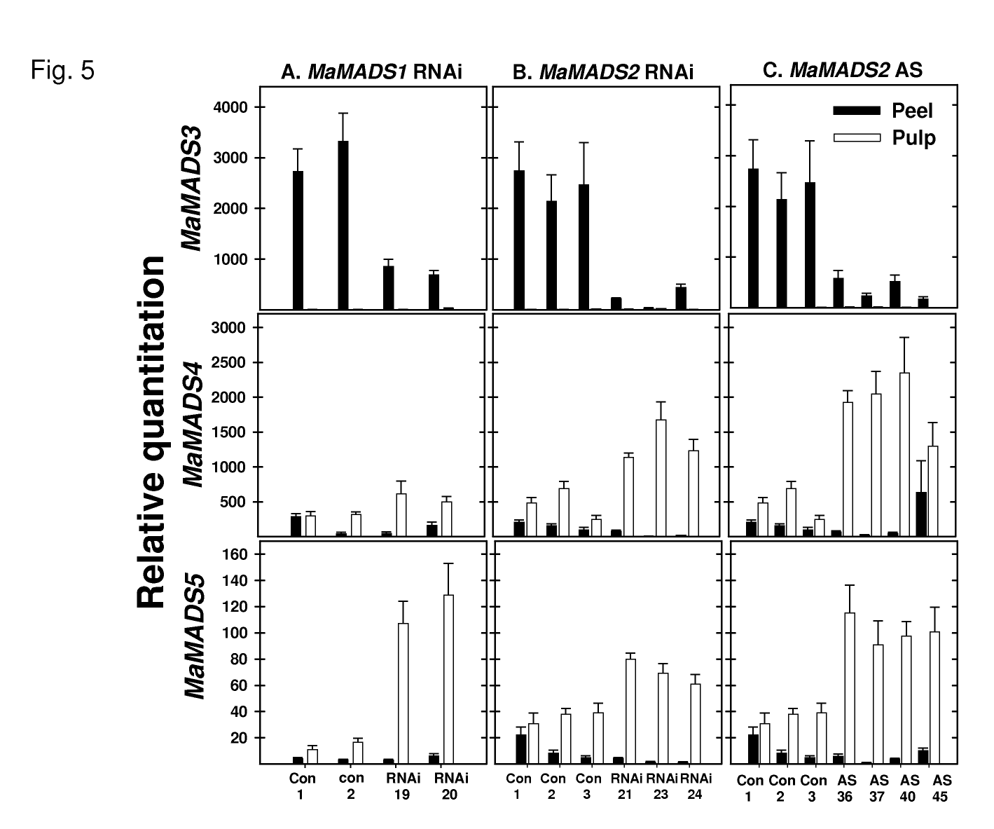

To study the possible interactions between MaMADS1 or MaMADS2 and other MaMADS genes isolated from fruit, the expression of MaMADS3, 4 and 5 have been determined in transgenic plants downregulated in either MaMADS1 (FIG. 5A) or MaMADS2 (FIG. 5B, C). The levels of MaMADS3 which was higher in peel than in pulp, was reduced in both types of transgenic plants in comparison to control. On the other hand, the levels of either MaMADS4 or MaMADS5 increased in the pulp of MaMADS2 knockdown plants. The levels of MaMADS4 and more so of MaMADS5 also increased in the pulp of MaMADS1 knockdown plants. These results indicate that both these genes are under a negative control of MaMADS2 and MaMADS1 in the pulp.

Chimera Production within Bunch Corroborate the Involvement of MaMADS1 in Fruit Ripening

Besides the banana bunches used for the above study, some of the bunches were not uniform in their color development after harvest and in some cases banana of fifth hand exhibited earlier ripening than banana from upper hands. To further elucidate this phenomenon, the DNA of banana from different hands of the same bunch of three different bunches were examined. It can be seen that in three plants that harbor the correct insert, judged by the PCR product of the leaf, only in two plants, bananas of the second hand had the insert, while those of the fifth hand did not have it. On the other hand, the banana of another positive plant (2-21) did not contain the insert. Hence, the transformation yielded chimera plants. These results were in agreement with ethylene and carbon dioxide production. In control fruits the ethylene production peak occurred on the 11th day after harvest in bananas from the second and the fifth hand. For fruit of the second hand which harbor the insert (plants 8 and 18), the appearance of ethylene and carbon dioxide peaks appeared 7 days later and in another plant (plant 21), which did not harbor the insert, those peaks appeared in a similar time frame to that of the control. Interestingly, the peaks of ethylene and carbon dioxide of fruits from the fifth hand which did not harbor the insert appeared between the seventh and the tenth day after harvest.

### Discussion

In this study we have established in banana that two of the fruit MaMADS-box genes are regulators of ripening. Reducing the transcript levels of these two genes reflected in reduced mRNA levels (FIG. 1), decreased ripening progression, as was demonstrated by inhibition of color change, delayed softening and slower accumulation of sugar in pulp and peel (FIG. 3). The fruits of the transgenic, once ripened, had similar characteristics to control fruits (FIG. 2, Table 5).

These two genes possibly act via inhibition of the climacteric respiration rise and associated ethylene production, since the patterns of CO₂ and ethylene production were altered in the transgenic fruit. Concerning climacteric respiration; in general, in MaMADS1 and in MaMADS2 RNAi fruits, the pattern of carbon dioxide production coincided with the pattern of ethylene production and the levels reached those in the controls. However, in transgenic fruit that did not produce any ethylene either from MaMADS1 RNAi or from MaMADS2 antisense, carbon dioxide production was delayed and increased concomitantly with the color change and then remained high. This observation indicates that although increased respiration might be affected by either MaMADS1 or MaMADS2, its initiation is independent of either of these genes.

The inhibition of ripening by reducing MaMADS1 levels was associated in one case with complete reduction of ethylene production; however in the other cases the ethylene peak was only delayed but eventually reached similar levels to those of control fruit (FIG. 2). In the case of MaMADS2 reduction, a small number of the transgenic fruit did not produce any ethylene (especially in the antisense manipulation); however the levels of ethylene in the remainder (either antisense or RNAi constructs) were not only delayed in their increase, but also reduced in terms of net levels achieved. Further support for the idea that MaMADS2 is a major regulator, stems from the fact that MaMADS2 negatively regulates the expression of MaMADS1, 4 and 5 in pulp and positively regulates MaMADS3 gene expression in the peel, since in the MaMADS2 RNAi or antisense transgenic fruits the levels of the former genes were increased and that of the last decreased (FIGS. 4, 5). In comparison, the levels of MaMADS2 and MaMADS4 genes expression in pulp were not affected in MaMADS1 transgenic fruit. Nevertheless, it seems that MaMADS1 negatively controls MaMADS5 gene expression in pulp and positively that of MaMADS3 in the peel. The increase in expression of these other negatively regulated MaMADS genes might explain why there is no complete inhibition of ripening as they may serve redundant functions. Thus, it is possible that with neither MaMADS1 nor MaMADS2, other genes may serve to execute the ripening process. Functional characterization of MaMADS 3, 4 and 5 remains to be demonstrated but will address this hypothesis once performed.

It is understood that the foregoing detailed description is given merely by way of illustration and that modifications and variations may be made therein without departing from the spirit and scope of the invention.

TABLE 1
Description of banana transgenic lines.

| | | | | PCR | |
|---|---|---|---|---|---|
| | Beginning of | Transfer | | positive | |
| Description | hardening | to field | Harvest | plants | Plants marks |
| MaMADS1 pK + C | 16/10/2007 | 1/4/2008 | 5/5/2009 | 29** | 4-19, 4-20 (2-8, |
| RNAi | | | | | 2-18, 2-21)*** |
| MaMADS2 C + | 6/9/2007 | 1/4/2008 | 2/6/2009 | 19 | 3-21, 3-23, 3-24 |
| 3′UTR RNAi | | | | | |
| MaMADS2 C + | 6/9/2007 | 1/4/2008 | 2/6/2009 | 2 | 3-45 |
| 3′UTR antisense* | 6/9/2007 | 1/4/2008 | 2/6/2009 | 16 | 3-36, 3-37, 3-40 |

*Two independent transformation were performed for this construct.
**3 plants died following their transfer to the field.
***In parenthesis are plants which exhibit chimera as described in FIG. 8.
Letters indicate the molecule sections used for transformation as described in Supplementary data, FIG. 1: C-full length of the C region; pK-partial length of the K region; 3′UTR-untranslated region at the 3′ end.

TABLE 2
Primers used for the creation of constructs.

| Description | | | Primers |
|---|---|---|---|
| MaMADS1 pK + | SEQ ID | FW | 5′-GGGGACAAGTTTGTACAAAAAA |
| C RNAi | NO: 7 | | GCAGGCTAAGGAATCTCCTTGGTGA |
| (pHELLSGATE2) | | | GGACTT-′3 |
| | SEQ ID | RV | 5′-GGGGACCACTTTGTACAAGAAA |
| | NO: 8 | | GCTGGGTAATCTGTGGAGTGGGTTG |
| | | | ACACTC-′3 |
| MaMADS2 C + | SEQ ID | FW | ′5-GGGGACAAGTTTGTACAAAAAA |
| 3′UTR RNAi | NO: 9 | | GCAGGCTGGAAACCAGGCCAAT CA |
| (pHELLSGATE8) | | | GCAACAA-′3 |
| | SEQ ID | RV | ′5-GGGGACCACTTTGTACAAGAAA |
| | NO: 10 | | GCTGGGTCGCAATCAT CAGCACAA |
| | | | GAAATAG-′3 |
| MaMADS2 | SEQ ID | FW | 5′-CTGCTCTCGAGCGCAATCATCA |
| C + 3′UTR | NO: 11 | | GCACAA-′3 |
| antisense | SEQ ID | RV | 5′-TGGCGGAATTCGGAAACCAGGC |
| (pBin117) | NO: 12 | | CAATCA-′3 |

Bold sequence in MaMADS2 antisense in pBin117 indicate the XhoI and EcoRI sites at the forward (FW) and reverse (RV) primers, respectively, and following these are MaMADS2 specific sequences. The bold sequences in primers for the creation of RNAi constructs depict the 25 attB1 and attB2 nucleotides at the forward (FW) and reverse (RV) primers, respectively. These sequences are followed by gene specific sequences.

TABLE 3
Primers used for verification of transgenic plants. Letters a-c
corresponding to the reactions described in Supplemental FIG., S2.

| | | Primers | |
|---|---|---|---|
| Reaction | Description | Forward | Reverse |
| a | MaMADS1 pK + C | SEQ ID NO: 13 | SEQ ID NO: 14 |
| | RNAi | 5′-ATCATTGATCTTACATTTGGATTG-′3 | 5′-GTCTCAGAAGAAGGTTGGAGGAGA-′3 |
| b | MaMADS2 C + | SEQ ID NO: 15 | SEQ ID NO: 16 |
| | 3′UTR RNAi | 5′-ATCATTGATCTTACATTTGGATTG-′3 | 5′-CAGGGTGACGGGTTCTTCCAA-′3 |
| c | MaMADS2 C + | SEQ ID NO: 17 | SEQ ID NO: 18 |
| | 3′UTR antisense | 5′-GTGGATTGATGTGACATCTCC-′3 | 5′-TGGCGGAATTCGGAAACCAGGCCAATCA-′3 |

TABLE 4
Primers used for determination of MaMADS transcript levels by Q-RT-PCR.
The specificity of the primers was determined in Elitzur et al, (2010).

| | Acession | Primers | |
|---|---|---|---|
| Gene | Num. | Forward | Reverse |
| MaMADS1 | EU869307 | SEQ ID NO: 19 | SEQ ID NO: 20 |
| | | 5′-ACAACTGGACATGTCACTGAAGG-′3 | 5′-GCTGGATGGGCACTGTTTTC-′3 |
| MaMADS2 | EU869306 | SEQ ID NO: 21 | SEQ ID NO: 22 |
| | | 5′-CAGGTGACGGGTTCTTCCAA-′3 | 5′-CGATTTGAAGAGTAGGTTCGCATT-′3 |
| MaMADS3 | EU869308 | SEQ ID NO: 23 | SEQ ID NO: 24 |
| | | 5′-TTGATCCTGGAGCAGATGGAA-′3 | 5′-GCTTTCAAGGTGGCACCTTCTA-′3 |
| MaMADS4 | EU869309 | SEQ ID NO: 25 | SEQ ID NO: 26 |
| | | 5′-TCCCAACACTCATGCTGTAGCT-′3 | 5′-CGCCATTTGATCTGGATGGT-′3 |
| MaMADS5 | EU8693010 | SEQ ID NO: 27 | SEQ ID NO: 28 |
| | | 5′-CCATTGTGGACGTCAATTCTCA-′3 | 5′-AAAGCGTCGCCCATCAAGT-′3 |

TABLE 5
Characterization of transgenic and non-transformed fruit.

| | | DAH | | TSS | TSS |
|---|---|---|---|---|---|
| Description | Plant mark | Color* | Firmness | Peel | Pulp |
| MaMADS1 pK + | 4-19 | 17.5 | 36.5 | 6 | 15 |
| C RNAi | 4-20 | 26 | 41 | 10 | 21 |
| MaMADS2 C + | 3-21, 3-23, 3-24 | 15-16 | 30.5 | 5 | 11-15 |
| 3′UTR RNAi | | | | | |
| MaMADS2 C + | 3-36, 3-37 | 18-19 | 20-31 | 3-4 | 10-15 |
| 3′UTR antisense | 3-40, 3-45 | 27 | 30 | 6-7 | 16 |
| Control | 1 | 14 | 28 | 9.5 | 20 |
| | 2 | 15 | 31 | 8 | 20 |
| | 3 | 12 | 28 | 6.5 | 14 |
| | 4 | 11 | 25 | 7.5 | 15.5 |
| | 5 | 12 | 28 | 7 | 17 |

DAH—days after harvest.
*The time till fruit reached a color of 100 determined as hue.
Firmness and TSS measurements are described in Materials and Methods.
Data were obtained from the FIG. 5.

We claim:

1. A method for producing a transgenic banana plant comprising:
a. providing a banana plant, banana plant tissue or banana plant cell which is capable of regeneration;
b. transforming said plant, plant tissue or cell with a DNA construct comprising a silencing nucleic acid sequence operatively linked to a promoter effective for expression in the fruit of said banana plant, wherein said silencing nucleic acid is effective for significantly reducing or eliminating the expression of MaMADS1 or MaMADS2 or both in said fruit; and
c. generating a transgenic plant from the transformed plant, plant tissue or plant cell.

2. The method of claim 1 further comprising selecting transgenic plants producing fruit which exhibits significantly delayed ripening in comparison to a non-transformed control plant.

3. The method of claim 1 wherein said banana plant is selected from the group consisting of the dessert banana and plantains.

4. The method of claim 1 wherein said silencing nucleic acid sequence is selected from the group consisting of an antisense RNA encoding nucleic acid sequence and an RNAi encoding nucleic acid sequence.

5. The method of claim 4 wherein said silencing nucleic acid sequence is selected from the group consisting of:
i) a nucleic acid sequence comprising at least 30 consecutive bases of the banana MaMADS1 or MaMADS2 gene;
ii) a nucleic acid sequence comprising at least 30 bases and having at least 80% homology to i);
iii) a nucleic acid sequence comprising at least 15 bases and which hybridizes under stringent conditions to the banana MaMADS1 or MaMADS2 gene.

6. The method of claim 5 wherein said silencing nucleic acid sequence is at least 75 bases.

7. The method of claim 5 wherein said silencing nucleic acid sequence is selected from the group consisting of:
i) a nucleic acid sequence comprising at least 30 consecutive bases of the C, I or K domain or the untranslated region of the banana MaMADS1 or MaMADS2 gene;
ii) a nucleic acid sequence comprising at least 30 bases and having at least 80% homology to i);
iii) a nucleic acid sequence comprising at least 15 bases and which hybridizes under stringent conditions to the C, I or K domain or the untranslated region of the banana MaMADS1 or MaMADS2 gene.

8. The method of claim 5 wherein said silencing nucleic acid sequence is selected from the group consisting of:
i) a nucleic acid sequence comprising at least 30 consecutive bases of the C domain or the untranslated region of the banana MaMADS1 or MaMADS2 gene;
ii) a nucleic acid sequence comprising at least 30 bases and having at least 80% homology to i);
iii) a nucleic acid sequence comprising at least 15 bases and which hybridizes under stringent conditions to the C domain or the untranslated region of the banana MaMADS1 or MaMADS2 gene.

9. The method of claim 5 wherein said MaMADS1 gene comprises SEQ ID NO: 1 and said MaMADS2 gene comprises SEQ ID NO:2.

10. A transgenic banana plant produced by the process of claim 1.

11. A transgenic banana plant produced by the process of claim 2.

12. Banana fruit of the transgenic banana plant of claim 10.

13. Banana fruit of the transgenic banana plant of claim 11.

14. A nucleic acid construct comprising a silencing nucleic acid sequence operatively linked to a promoter effective for expression in the fruit of a banana plant, wherein said silencing nucleic acid is effective for significantly reducing or eliminating the expression of MaMADS1 or MaMADS2 or both in said fruit.

15. The nucleic acid construct of claim 14 wherein said silencing nucleic acid sequence is selected from the group consisting of an antisense DNA encoding nucleic acid sequence and an RNAi encoding nucleic acid sequence.

16. The nucleic acid construct of claim 15 wherein said silencing nucleic acid sequence is selected from the group consisting of:
i) a nucleic acid sequence comprising at least 30 consecutive bases of the banana MaMADS1 or MaMADS2 gene;
ii) a nucleic acid sequence comprising at least 30 bases and having at least 80% homology to i);
iii) a nucleic acid sequence comprising at least 15 bases and which hybridizes under stringent conditions to the banana MaMADS1 or MaMADS2 gene.

17. The nucleic acid construct of claim 16 wherein said silencing nucleic acid sequence is at least 75 bases.

18. The nucleic acid construct of claim 16 wherein said silencing nucleic acid sequence is selected from the group consisting of:
i) a nucleic acid sequence comprising at least 30 consecutive bases of the C, I or K domain or the untranslated region of the banana MaMADS1 or MaMADS2 gene;
ii) a nucleic acid sequence comprising at least 30 bases and having at least 80% homology to i);
iii) a nucleic acid sequence comprising at least 15 bases and which hybridizes under stringent conditions to the C, I or K domain or the untranslated region of the banana MaMADS1 or MaMADS2 gene.

19. The nucleic acid construct of claim 16 wherein said silencing nucleic acid sequence is selected from the group consisting of:
i) a nucleic acid sequence comprising at least 30 consecutive bases of the C domain or the untranslated region of the banana MaMADS1 or MaMADS2 gene;
ii) a nucleic acid sequence comprising at least 30 bases and having at least 80% homology to i);
iii) a nucleic acid sequence comprising at least 15 bases and which hybridizes under stringent conditions to the C domain or the untranslated region of the banana MaMADS1 or MaMADS2 gene.

20. The nucleic acid construct of claim 16 wherein said MaMADS1 gene comprises SEQ ID NO: 1 and said MaMADS2 gene comprises SEQ ID NO:2.
